(19) United States
(12) Patent Application Publication
(10) Pub. No.: US 2007/0043329 A1
(43) Pub. Date: Feb. 22, 2007

(54) Male incontinence briefs

(76) Inventor: Barbara Evans, Santa Barbara, CA (US)

Correspondence Address: Michael G. Petit, P.O. Box 91929, Santa Barbara, CA, 93190-1929, US

(21) Appl. No.: 11/207,372

(22) Filed: Aug. 18, 2005

(51) Int. Cl. A61F 5/44

(52) U.S. Cl. 604/349; 604/385.09

(57) ABSTRACT

Incontinence briefs having a resealable, substantially leak-proof and resealable flap in a front section thereof. The resealable flap opens from the bottom upwardly to provide access to a wearer's penis. The construction permits the wearer to comfortably urinate into a portable urinal without the necessity of removing the garment. The garment is particularly adapted for males. The construction of the resealable flap enables a wearer to urinate without bending the penis thereby enabling more complete emptying of the bladder. In addition, an expandable fluid-impermeable membrane on the lateral edges of the flap reduces leakage from the garment when the flap is closed. The briefs are suitable for males of all ages.

### BACKGROUND OF THE INVENTION

1. Field of the Invention

The present invention relates to an incontinence garment and, more particularly, to absorbent briefs having a resealable flap on a front portion thereof disposed anterior to the penis of a wearer.

2. Prior Art

Diapers and incontinence briefs operable for preventing the leakage of urine from an incontinent wearer are well known in the art. Many “incontinent” patients have some degree of control over their urethral sphincter and can urinate into a urinal if such a urine receptacle can be conveniently accessed without undue delay. Accordingly, incontinence briefs have been developed for males that incorporate an access flap in a front portion thereof through which the penis can be accessed without removing the briefs.

Berke, in U.S. Pat. No. 5,984,910, discloses a urinary incontinence device for use by a male that includes an undergarment worn closely to the body and including an opening or hole through which the penis passes and is kept upright and close to the body by a flap across the opener in the undergarment. The flap may include an occlusion device such as an inflatable cuff integrally incorporated therein and aligned with the opening to be positioned between the base of the shaft of the penis and the scrotum to occlude the urethral passage of the penis. The occlusion device is designed to apply sufficient pressure to occlude the urethral passage without cutting off circulation of blood within the penis through the dorsal vein. The flap, which opens from the top, is aledged to retain the penis in a position operable for occlusion of the urethral passage to prevent urine discharge into the incontinence briefs.

Pudlo, in U.S. Pat. No. 5,618,279, discloses a protection device for use with an incontinence garment that includes a generally planar deflection shield formed of a moisture impervious material. The deflection shield is of sufficient length to extend from above the penis shaft downwardly to the approximate level of the scrotum of the male. The deflection shield includes a clearance aperture permitting the penis shaft to extend through the deflection shield to assure urine is deflected away from the scrotum of the male. The deflection shield includes a sack-like structure extending from a lower portion of the deflection shield adapted to at least partially enclose the scrotum of the male. In an alternative embodiment of the invention, a lower portion of the deflection shield is directly attached to the incontinence garment. Again, as with Burke, the deflection shield opens from the top.

Wyant, in U.S. Pat. No. 5,843,065, discloses an incontinence diaper having a front section, a back section and a crotch section joining the front and back panels. Fasteners are provided on the upper corners of the front and rear sections for use in joining them together to don the diaper. An absorbent pad is detachably mounted on the inner surface of the front section in the male genital covering area to absorb involuntary urinary discharge. An opening is provided in the front section of the diaper in the male genital covering area. The opening is normally covered by the pad. Moving the pad to uncover the opening allows the diaper wearer to normally urinate through the opening. The opening is preferably shaped to allow a portion of the absorbent pad to be pulled out through the opening to be out of the way during urination. The construction does not include a resealable flap.

Casale, in U.S. Pat. No. 4,944,733, discloses a diaper/incontinent device for children or adults. The diaper comprises a front portion and a back portion connected by a crotch portion, the crotch portion includes leg openings which allow the diaper to be attached around the legs and waist of the user. The front portion of the diaper further includes a reclosable penile opening which extends from the waist opening through the center of the front portion down into the crotch portion of the diaper. The opening is held closed by tape fastener or the like. The penile opening comprises a pair of flaps which overlap each other in a closed position and which can be pulled apart to allow the user to urinate therethrough in an open position. The opening can then be reclosed and/or resealed. The penile opening can be advantageously formed without otherwise disturbing the secure attachment of the diaper around the user's legs and waist. The diaper, while absorbent and permitting the wearer to use a urinal or toilet in the conventional manner, includes a flap that opens from the top and does not provide sealing means around the periphery of the flap for preventing the leakage of urine from within.

The normal, most comfortable position for the penis when a male is urinating while either standing or recumbant is for the penis to be oriented downwardly or toward the feet. If, during urination, there is a bend in the penis, the result is difficult urination, retention of urine within the bladder or even painful urination. If a male is wearing an incontinence brief fitted with a flap and the flap opens from the top, the penis must be bent downward over the flap's lower “hinge” portion to direct a stream of urine into a receptacle. If, however, the flap is hinged at the top and opens from the bottom, the penis can be directed toward a urine receptacle with minimal bending. Further, when the flap is closed, it is desirable that there is minimal leakage of urine from the absorbent interior of the briefs through the periphery of the flap. Accordingly, there is a need for an incontinence brief having a flap for accessing the penis that reduces bending of the penis during urination and minimizes leakage of urine from within the brief in the event that the wearer urinates in the briefs.

### SUMMARY

The present invention is directed to a male incontinence brief that substantially obviates one or more of the limitations of the related art. To achieve these and other advantages and in accordance with the purpose of the invention, as embodied and broadly described herein, the invention includes a incontinence brief with a forwardly disposed resealable flap that opens downwardly and includes urine retaining means disposed around the lateral edges of the flap operable for preventing leakage of urine from within the brief. The incontinence brief is designed to be worn by an incontinent male. The brief is operable for controlling the leakage of urine therefrom while providing external access to the penis.

The male incontinence brief comprises an absorbant undergarment having a pair of leg openings in a lower portion thereof, a waist opening in an upper portion thereof, and a penis access hole disposed in a front portion of the undergarment for receiving and externally accessing the penis. The absorbant undergarment further comprises a resealable flap having: (i) a hinged end nonreleasably attached to the undergarment above the penis access hole; (ii) a closure end disposed in opposition to the hinged end, the closure end being adapted to be detachably connected to the undergarment; and (iii) lateral edges therebetween. A sheet of extendable fluid-impermeable material is disposed between each of the lateral edges of the flap and an outer surface of the undergarment to provide a fluid-impermeable seal therebetween. The flap on the male incontinence brief preferably includes an apertured sheet of fluid-impermeable material interposed between, and affixed to, the lower edge of the sheets of extendable fluid-impermeable material and the outer surface of the undergarment. The apertured sheet is then affixed to the outer surface of the undergarment such that the aperture therein overlies the penis access opening in the undergarment. The flap preferably further includes a flap cover having a sheet of absorbant material attached thereto to substantially occlude the penis access hole when said flap is in a closed position.

The features of the invention believed to be novel are set forth with particularity in the appended claims. However the invention itself, both as to organization and method of operation, together with further objects and advantages thereof may be best understood by reference to the following description taken in conjunction with the accompanying drawings.

### BRIEF DESCRIPTION OF THE DRAWINGS

FIG. 1 is a front view of the incontinence brief in accordance with the present invention showing the disposition of the resealable flap, closed in FIG. 1, with respect to the penis of a male wearer.

FIG. 2 is a front view of the incontinence brief in accordance with FIG. 1 showing the resealable flap illustrating the resealable flap in an open 7 position.

FIG. 3 is a side view of the incontinence brief of FIGS. 1 and 2 showing the flap in an open position and a urinal (in phantom) positioned to receive urine from the wearer.

FIG. 4 is a top view of the incontinence briefs of FIGS. 1 and 2 with the resealable flap open and showing the leg openings.

FIG. 5 is an enlarged perspective view of the resealable flap in a partially open position.

FIG. 6 is a sectional view of the outer, forward portion of the flap of FIG. 5 viewed from the lower surface thereof along section plane 6-6 of FIG. 5.

### DESCRIPTION OF THE PREFERRED EMBODIMENTS

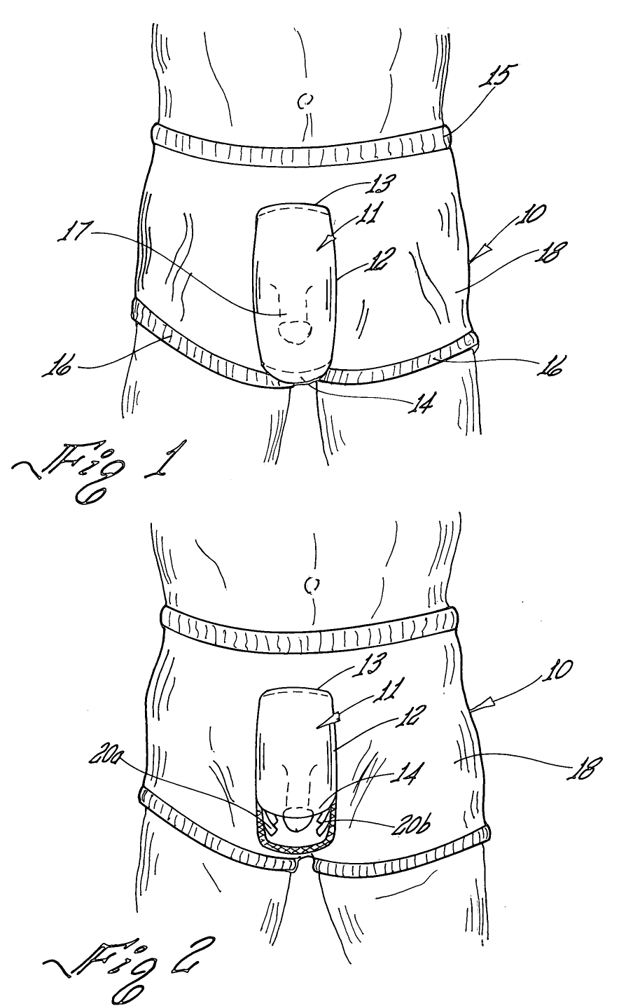

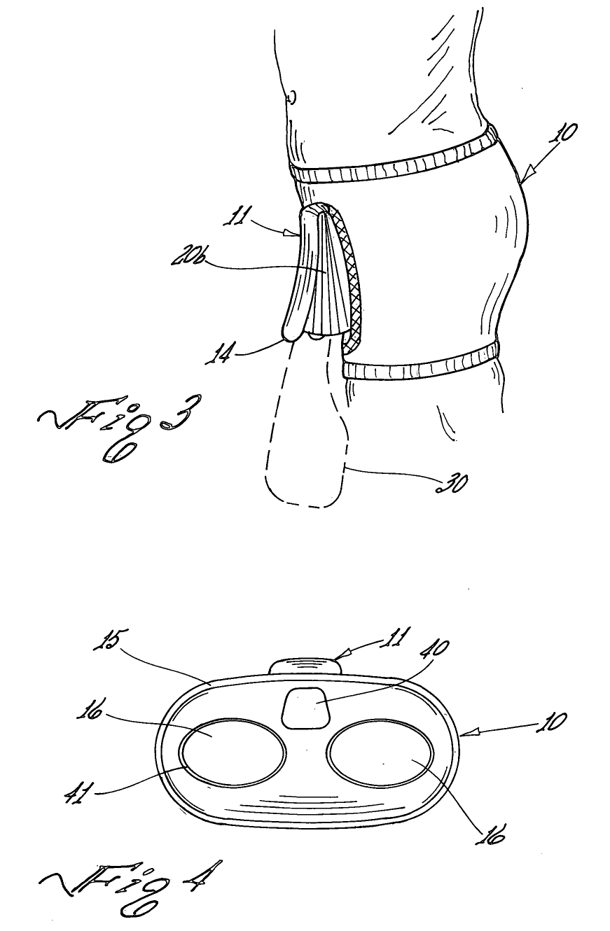

Turning now to FIG. 1, incontinence briefs in accordance with the present invention is generally indicated in front view at numeral 10. The briefs 10 include a resealable flap 11 having a lateral edge 12, a upper hinge portion 13 and a lower closure portion 14. The incontinence briefs 10 further include a waist portion 15 and leg openings 16. The waist portion 15 and leg openings 16 preferably include an elastic member operable for forming a tight, leak-proof engagement with the portions of the wearer's body in contact therewith. The position of the wearer's penis is shown in phantom at 17. The outer surface 18 of the incontinence briefs 10 preferable comprises a fluid-impermeable material. FIG. 2 is a front, somewhat perspective view of the incontinence brief 10 of FIG. 1 illustrating the resealable flap 11 in an open position. The lateral edges 12 of the flap are maintained in leak-proof engagement with the outer surface 18 of the briefs 10 by means of a leak-proof material 20a and 20b disposed therebetween. The lower surface of the closure portion 14 of the flap 11 is preferably coated with a releasable adhesive or bears a strip of a hook or loop fastener that matingly engages a strip of loop or hook fastener adhered to the portion of the outer surface 18 of the briefs that is in juxtaposition with the closure portion 14 when the flap 11 is closed. FIG. 3 is a side view of the incontinence brief 10 of FIGS. 1 and 2 showing the flap 11 in an open position and a urinal 30 (in phantom) positioned to receive urine from the wearer.

FIG. 4 is a top view of the incontinence briefs 10 of FIGS. 1-3 with the resealable flap 11 open and showing the leg openings 16. An opening 40 in the briefs provides means for the wearer's penis to be externally accessed when the flap 11 is in the open position. As mentioned earlier, the waist 15 and the edges 41 of the leg openings 16 preferably comprise an elastic member in order to insure that the periphery of the respective openings is in intimate contact with the wearer's body to prevent leakage of urine from an absorbant material (disposed within the brief 10 between a fluid-permeable inner skin-contacting layer of fabric and a fluid impermeable (usually plastic) outer layer in accordance with the prior art) to the exterior (outer) surface 18 of the briefs 10.

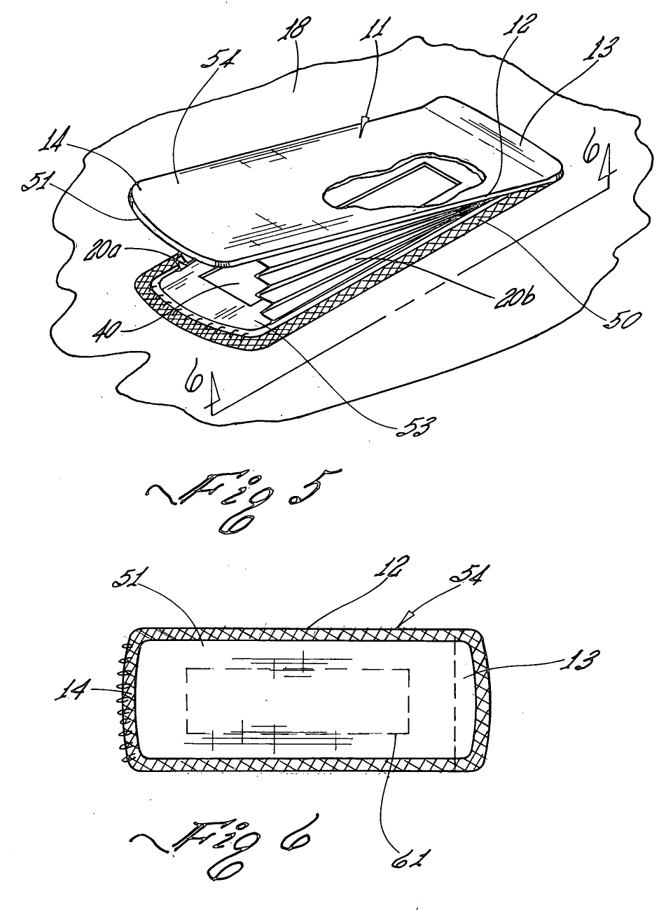

The resealable flap 11 is illustrated in enlarged, partially cutaway perspective view in FIG. 5. The resealable flap 11 can be made as a separate article and adhered to the briefs to overlie the penis access opening 40. If the flap 11 is made as a separate article to be affixed to the briefs during the manufacturing process, an adhesive strip 50 is applied to the briefs to engage a peripheral bottom edge of the expandable leak-proof material 20a and 20b thereby forming a leak-proof seal between the flap 11 and the outer surface 18 of the briefs 10. The flap 11 may alternatively include a sheet 53 having a rectangular cutout therein that overlies the elongate penis access opening 40 in the briefs. In this embodiment, the lower edge of the expandable fluid-impermeable material 20a and 20b is affixed to the top surface of sheet 53 and the top edge of the expandable fluid-impermeable material 20a and 20b is affixed to the lower surface 51 of a flap cover 54. The sheet 53 is then adhered to the outer surface 18 of the briefs such that the rectangular cutout in the sheet 53 overlies the penis access opening 40 in the briefs. The expandable leak-proof material 20a and 20b may comprise sheets of accordion-folded plastic as shown in FIG. 5, or it can comprise sheets of an elastically stretchable material such as latex or silicone sheeting. The upper edge of the expandable leak-proof material 20a and 20b is affixed in fluid-sealing engagement with the lower surface of the flap cover 54 and the lower edge is similarly affixed to the upper surface of the sheet 53. The lower, opposing surface of sheet 53 is affixed in fluid-sealing engagement with the outer surface 18 of the briefs by affixation means such as welding, solvent fusion or adhesives.

With reference now to FIG. 6, the lower surface of the flap cover 54 is presented as viewed along viewing plane 6-6 of FIG. 5. The adhesive strip 51 adheres to the outer surface of the garment in the hinge portion 13 and to the upper edge of the expandable leak-proof material 20a and 20b along the lateral edges 12. The closure portion 14 of the flap cover has a releasable closure means such as hook or loop fasteners or a releasable adhesive thereon that engages mating closure means disposed on the portion of the upper surface of sheet 53 that is in juxtaposition with the closure portion 14 when the flap 11 is closed. In addition, an absorbant pad 61 (shown in phantom in FIG. 6) may be adhered to the lower surface of the flap cover 54 to substantially occlude the penis access opening 40 when the flap is closed.

While particular embodiments of the present invention have been illustrated and described, it would be obvious to those skilled in the art that various other changes and modifications can be made without departing from the spirit and scope of the invention. For example, the briefs can be worn by a male of any age. It is therefore intended to cover in the appended claims all such changes and modifications that are within the scope of this invention.

What I claim is:

1. A male incontinence brief to be worn by an incontinent person, said brief being operable for controlling the leakage of urine therefrom, said male incontinence brief comprising:
(a) an absorbant undergarment having a pair of leg openings in a lower portion thereof, a waist opening in an upper portion thereof, and a penis access hole disposed in a front portion of the undergarment for receiving and externally accessing the penis;
(b) a resealable flap having: (i) a hinged end nonreleasably attached to the undergarment above said penis access hole; (ii) and a closure end disposed in opposition to said hinged end, said closure end being adapted to be detachably connected to said undergarment; and (iii) lateral edges therebetween;
(c) a sheet of extendable fluid-impermeable material disposed between each of said lateral edges of said flap and an outer surface of said undergarment to make a fluid-impermeable seal therebetween.

2. The male incontinence brief according to claim 1 wherein an apertured sheet of fluid-impermeable material is interposed between said sheet of extendable fluid-impermeable material and said outer surface of said undergarment and is affixed thereto.

3. The male incontinence brief according to claim 1 wherein said flap further comprises a flap cover having a sheet of absorbant material attached thereto to substantially occlude said penis access hole when said flap is in a closed position.

4. The male incontinence brief according to claim 2 wherein said flap further comprises a flap cover having a sheet of absorbant material attached thereto to substantially occlude said penis access hole when said flap is in a closed position.
